(19) United States
(12) Patent Application Publication
(10) Pub. No.: US 2024/0138684 A1
(43) Pub. Date: May 2, 2024

(54) SYSTEM AND METHOD FOR MECHANICAL CHARACTERIZATION OF HETEROGENEOUS TISSUE

(71) Applicant: Massachusetts Institute of Technology, Cambridge, MA (US)

(72) Inventors: Bharath NARAYANAN, Renens (CH); Max Louis OLENDER, Cambridge, MA (US); Farhad Rikhtegar NEZAMI, Lexington, MA (US); David MARLEVI, Stockholm (SE); Elazer R. EDELMAN, Brookline, MA (US)

(21) Appl. No.: 18/277,128

(22) PCT Filed: Feb. 15, 2022
(86) PCT No.: PCT/US2022/016420

§ 371 Date: Aug. 14, 2023

Related U.S. Application Data
(60) Provisional application No. 63/149,437, filed on Feb. 15, 2021.

(51) Int. Cl. A61B 5/02; A61B 5/00; G16H 30/40; G16H 50/20

(52) U.S. Cl. CPC A61B 5/02007; A61B 5/0066; G16H 30/40; G16H 50/20

(57) ABSTRACT

A method for determining material properties of a plurality of tissues in a subject includes receiving a first sequence of cardiovascular images of a region of interest of the subject and a first set of pressure data associated with the first sequence of cardiovascular images and receiving a second sequence of cardiovascular images of the region of interest of the subject and a second set of pressure data associated with the second sequence of cardiovascular images. The method can further include transforming, using a processor, the first sequence of cardiovascular images to a first three-dimensional (3D) finite element (FE) mesh with heterogeneous material distribution, transforming, using the processor, the second sequence of cardiovascular images to a second 3D FE mesh with heterogeneous material distribution, and performing, using the processor, an iterative optimization process on the first and second 3D FE meshes to determine one or more material properties of at least one of the plurality of tissues.

### CROSS-REFERENCE TO RELATED APPLICATIONS

This application is based on, claims priority to, and incorporates herein by reference in its entirety U.S. Ser. No. 63/149,437 filed Feb. 15, 2021, and entitled “A Method Leveraging Morphology for Non-Destructive In Situ Mechanical Characterization of Heterogeneous Biological Tissue.”

### BACKGROUND

Geometry, structure, and material constitutive properties together describe the mechanical behavior of a physical system. All three of these parameters are uniquely altered for diseased arteries. If known, this information can provide unique insights into the pathophysiological state of an assessed vascular structure. In particular, computational modeling and simulation offer qualitative and quantitative windows into disease states, enabling their use in clinical prognostication, treatment planning, device design, and establishment of intervention guidelines. However, without a sound understanding of the patient-specific mechanics of a system, the function, risk profile, and mechanisms of diseased vessels, devices, and interventions cannot be fully appreciated.

Stress is one such important driving mechanical factor. Coupled to stress state are material constitutive properties, which describe the strength of materials and their response to loading. While these properties can be inferred, to an extent, from a general composition of tissue, material properties vary dramatically between patients as well as among the same class of tissue or plaque. A robust body of work has demonstrated the fundamental impact of material property on stress values and distribution in modeled arteries, which has motivated efforts to identify appropriate properties to use in such computational models. Therefore, an important area of investigation has been the determination of mechanical properties of tissue in situ.

The directly observable state which provides insight into stress and mechanical properties is displacement, or strain. Specifically, given a measured strain, either loading condition (stress) or material properties can be determined if the other is known. In the field of solid mechanics, this basic principle is exploited in determining constitutive mechanical properties of a material through tensile testing. A known load is applied to a material shaped into a known geometry, and the material displacement is measured. From these results, mechanical properties can be determined. This type of mechanical testing has been performed on excised tissue through the separation of various plaque components and tissue classes. Unfortunately, such controlled tests cannot be performed non-destructively, and are, naturally, not feasible for living tissue in situ. Computational methods offer some alternative to determining stress distribution within a structure or constitutive properties of the composition.

Based on the basic principle of tensile testing, these computational approaches estimate governing constitutive relationships by observing the displacement response of tissue to a distorting force. One approach leveraging this coupling is inverse finite element (FE) analysis, where FE modeling is used to iteratively simulate the imposed loading, updating material properties with each iteration until the simulation results match the observed outcome. Several prior methods relied on accurate acquisition of displacement/strain fields to serve as the observable outcome. However, experimental displacement data are often restricted to 2D in-plane observations and susceptible to low signal-to-noise ratio. More recent methods have forgone displacement fields in favor of matching overall geometry at discrete states. One prior method uses the inner and outer vessel diameters of a 3D mesh as the observed outcomes to be matched at multiple loading points, while another prior method treats the entire inner and outer surfaces of the 3D meshes as the object of comparison. These surface matching approaches are used to fit single-parameter models representing multiple tissue types or a nonlinear multi-parameter material model for a single homogenized vessel wall. Current approaches, however, are limited to either homogenized or simplified material representations.

Prior two-dimensional (2D) inverse methods are limited by their inability to capture the complex multi-dimensional linked stress-strain relationships in the cardiovascular system due to the use of single parameter constitutive models. The 2D methods typically cannot incorporate out-of-plane stresses, neglecting effects of physiologic undulations of the vessel wall and the 3D motion of the heart itself. Single-parameter models are commonly employed to avoid over-parameterization and ensure solution uniqueness when using displacement data derived from images obtained at two distinct intravascular pressures. To enable recovery of higher-parameter material models, the methods may acquire data at several intravascular pressures, as previously done in 2D and 3D. However, increasing the number of required images limits clinical translation.

It would be desirable to provide systems and methods for mechanical characterization of heterogeneous tissue that are configured to account for material heterogeneity and to enable recovery of multiple patient-specific parameters for multiple tissues using only two sets of in vivo image acquisition.

### SUMMARY OF THE DISCLOSURE

In accordance with an embodiment, a method for determining material properties of a plurality of tissues in a subject includes receiving a first sequence of cardiovascular images of a region of interest of the subject and a first set of pressure data associated with the first sequence of cardiovascular images and receiving a second sequence of cardiovascular images of the region of interest of the subject and a second set of pressure data associated with the second sequence of cardiovascular images. The region of interest can include a plurality of tissues. The method can further include transforming, using a processor, the first sequence of cardiovascular images to a first three-dimensional (3D) finite element (FE) mesh with heterogeneous material distribution, transforming, using the processor, the second sequence of cardiovascular images to a second 3D FE mesh with heterogeneous material distribution, and performing, using the processor, an iterative optimization process on the first and second 3D FE meshes to determine one or more material properties of at least one of the plurality of tissues. The iterative optimization process can utilize interfaces between the plurality of tissues.

In accordance with another embodiment, a system for determining material properties of a plurality of tissues in a subject includes an input configured to receive a first sequence of cardiovascular images of a region of interest of the subject and a first set of pressure data associated with the first sequence of cardiovascular images. The input can also be configured to receive a second sequence of cardiovascular images of the region of interest of the subject and a second set of pressure data associated with the second sequence of cardiovascular images. The region of interest can include a plurality of tissues. The system further includes a pre-processing module configured to transform the first sequence of cardiovascular images to a first three-dimensional (3D) finite element (FE) mesh with heterogeneous material distribution. The pre-processing module can also be configured to transform the second sequence of cardiovascular images to a second 3D FE mesh with heterogeneous material distribution. The system further includes an optimizer configured to perform an iterative optimization process on the first and second 3D FE meshes to determine one or more material properties of at least one of the plurality of tissues. The iterative optimization process can utilize interfaces between the plurality of tissues.

### BRIEF DESCRIPTION OF THE DRAWINGS

The present invention will hereafter be described with reference to the accompanying drawings, wherein like reference numerals denote like elements.

FIG. 1 is a block diagram of a system for determining material properties of a plurality of tissues in a subject in accordance with an embodiment;

FIG. 2 illustrates a method for determining material properties of a plurality of tissues in a subject in accordance with an embodiment;

FIG. 3 illustrates a method for generating a three-dimensional (3D) finite element (FE) mesh based on a set of cardiovascular images of a subject in accordance with an embodiment;

FIG. 4 illustrates an optimization method using an inverse 3D FE process in accordance with an embodiment;

FIG. 5 is a diagram illustrating an example difference operator quantifying multiple feature changes for use in an objective function of the optimization method in FIG. 4 in accordance with an embodiment; and

FIG. 6 is a block diagram of an example computer system in accordance with an embodiment.

### DETAILED DESCRIPTION

The present disclosure describes systems and methods for determining material properties (or parameters) of a plurality of tissues in a subject. The disclosed systems and methods can account for tissue heterogeneity and material nonlinearity in the recovery of constitutive behavior using imaging data acquired at different intravascular pressures, and incorporating interfaces between various tissue types (e.g., intra-plaque tissue types) into an objective function. Advantageously, the mechanical characterization technique can enable high-fidelity material parameter recovery for use in complex cardiovascular computational studies. In some embodiments, the material properties may be, for example, linear elastic material properties or nonlinear hyperelestic material properties. In some embodiments, the material models used to define nonlinear hyperelastic material properties may include, for example, an isotropic hyperelastic model or an anisotropic model that considers fiber orientation within the arterial wall.

In some embodiments, the described mechanical characterization technique can include an inverse finite element (FE) technique that may be configured to obtain multiple patient-specific material properties (or parameters) for several tissue types (e.g., 3D lesion components) using only two sets of in vivo image acquisitions. The multi-parameter recovery may be facilitated by incorporating micro-morphological information in the form of interfaces between tissues (e.g., intra-plaque interfaces) into an objective function. Accordingly, the system and method can provide inverse material characterization that utilizes an interface-matching approach. In some embodiments, the two sets of images may be acquired using high-resolution intravascular imaging techniques such as, for example, optical coherence tomography (OCT) or intravascular ultrasound (IVUS). Advantageously, multi-parameter, multi-component material characterization may enable patient-specific clinical interventions that take into account, for example, the unique mechanistic state that each diseased vessel presents.

In some embodiments, the disclosed system and method may be used to recover the material properties (or parameters) of multiple arterial plaque components using two sets of intravascular imaging data acquired at different intraluminal pressures. A pre-processing step may be used to first convert in vivo images into 3D FE geometries with heterogeneous material distribution. The 3D FE geometries may then be utilized by the described inverse FE technique to recover material properties that reproduce the behavior manifested by the two imaged states. In addition, in some embodiments, the described system and method is configured to recover multiple parameters for multiple materials through the incorporation of micro-morphological information in the form of tissue interfaces (e.g., intra-plaque tissue interfaces) into an objective function. Accordingly, the mechanical characterization method can advantageously be configured to leverage micro-morphological information (e.g., the micromorphology of the vessel wall including, for example, all intra-plaque interfaces) which can result in superior performance. Prior methods typically use only macro-morphological information, for example, in some prior methods the inner and outer vessel wall surfaces or the minimum and maximum diameters at a number of slices are compared.

Advantageously, the disclosed mechanical characterization system and method may be implemented in a clinical setting as it requires only two image acquisitions (e.g., two pullbacks) to recover an increased number of material parameters. By using intravascular imaging (e.g., OCT), and requiring only 2 pullbacks, in some embodiments the mechanical characterization method can provide information on, for example, the extant mechanical state of a diseased artery at a resolution of a few μm without a significant increase in patient risk. In doing so, the disclosed mechanical characterization method can help better inform clinicians and researchers on the patient-specific impact of different clinical interventions. In addition, by using 3D FE models, the disclosed system and method not only captures 3D morphological information, but can also ensure that the applied loads are physiologically representative, for example, by accounting for out-of-plane stresses and loads.

In some embodiments, the described mechanical characterization system and method (and inverse FE framework) may be used in clinical applications where lesion-specific mechanical characterization can help better inform clinical scientists and interventionalists of the potential impact of certain morphological phenotypes on different clinical interventions. For example, the mechanical characterization method may allow a view into the biomechanical state of, for example, atherosclerotic arteries and provide expanded awareness of the mechanical context of interventions, offering a more comprehensive assessment to guide clinical decision-making in addition to improving patient-specific models of individual artery segments. In allowing for the recovery of more accurate and physiologically relevant material parameters, it can also facilitate high-fidelity computational studies which are imperative for evaluating the lesion-dependent impact of novel and legacy interventional devices and processes. Advantageously, the mechanical characterization system and method may allow for the realization of tangible benefits for the care of patients with cardiovascular disease.

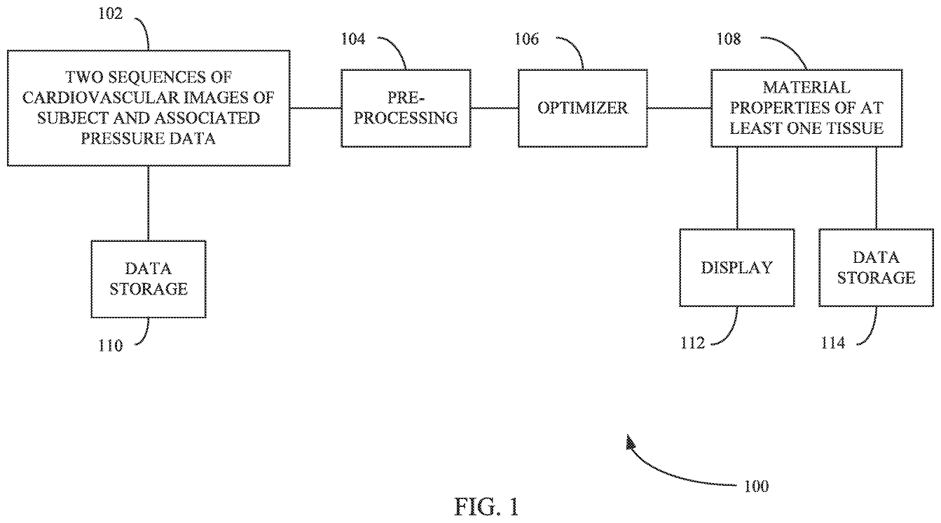

FIG. 1 is a block diagram of a system for determining material properties of a plurality of tissues in a subject in accordance with an embodiment. The system 100 can include an input of two sequences of cardiovascular images of a region of interest of a subject and associated pressure data 102, a pre-processing module 104, an optimizer 106, an output of at least one material property for at least one tissue in the region of interest 108, data storage 110, a display 112, and data storage 114. Each of the two sequences of cardiovascular images are acquired from a region of interest that includes a plurality of tissues. For example, in some embodiments, the plurality of tissues may include tissue types such as artery, fibrous, lipid, calcified, mixed, and healthy wall tissue. The two sequences of cardiovascular images may be acquired using known cardiovascular imaging techniques. In some embodiments, each of the two sequences of cardiovascular images can be acquired using intravascular imaging techniques, for example, optical coherence tomography (OCT) and intravascular ultrasound (IVUS), and acquired using known imaging systems (e.g., OCT or IVUS imaging systems). For intravascular imaging techniques (e.g., OCT or IVUS), each sequence of cardiovascular images may be acquired using, for example, a pullback or tomographic acquisition. In some embodiments, each of the two sequences of cardiovascular images may be acquired using imaging techniques such as magnetic resonance imaging (MRI) or computed tomography (CT), and acquired using known imaging systems (e.g., MRI or CT imaging systems). In some embodiments, the same cardiovascular imaging technique may be used to acquire both of the sequences of cardiovascular images. In some embodiments, one of the two sequences of cardiovascular images can be acquired using a first cardiovascular imaging technique and the other of the two sequences of cardiovascular images can be acquired using a second, different cardiovascular imaging technique. While the following description will be discussed with reference to intravascular imaging and intravascular imaging systems, it should be understood that the systems and methods described herein may be used with other types of cardiovascular imaging techniques.

Each of the two sequences of cardiovascular images can provide, for example, three-dimensional (3D) information on spatial tissue distribution. In addition, each sequence of cardiovascular images corresponds to or is associated with a set or pressure data, for example, each sequence of cardiovascular images may be acquired at a constant or varying intraluminal pressure that can be measured during the image acquisition. Each of the two sequences of cardiovascular images can be acquired at different pressure(s). In some embodiments the first and second set of pressure data may be measured using a manometer. In some embodiments, the pressure data may be recorded by the imaging system or apparatus used to acquire the sequences of cardiovascular images, for example, an imaging catheter with a built-in pressure transducer, or an imaging catheter that includes materials of known stiffness such that the pressure can be inferred from the resulting image by viewing the displacement of the known material shown in the image. In some embodiments, the pressure may be estimated from a contrast or flush injector used during the imaging process, for example, liquid contrast/flush can be injected into the arteries to clear blood from the field of view during intravascular OCT imaging. In such embodiments, the contrast or flush injector may be used to specify and/or control the pressure rather than just be passively observed. In some embodiments, the two sequences of cardiovascular image and the associated set of pressure data for each sequence of cardiovascular images 102 may be retrieved from data storage (or memory) 110 of the system 100, data storage of a imaging system, for example, an OCT imaging system, or data storage of other computer systems.

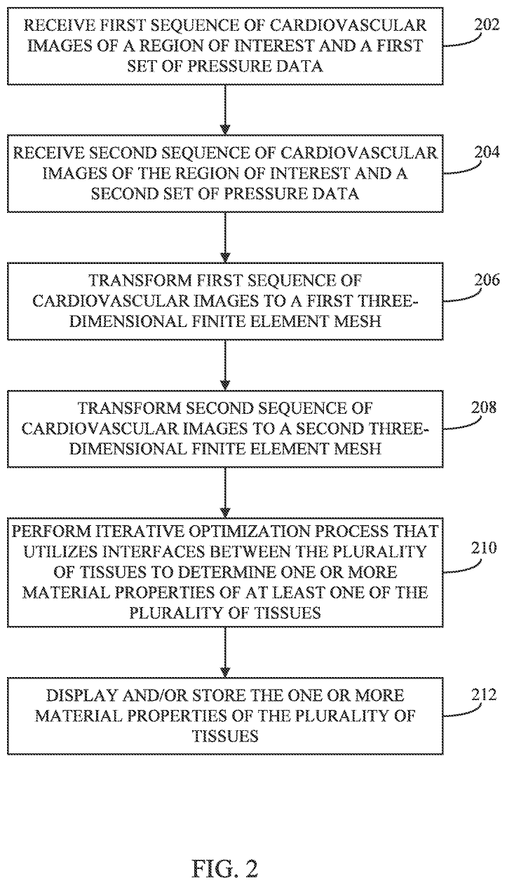

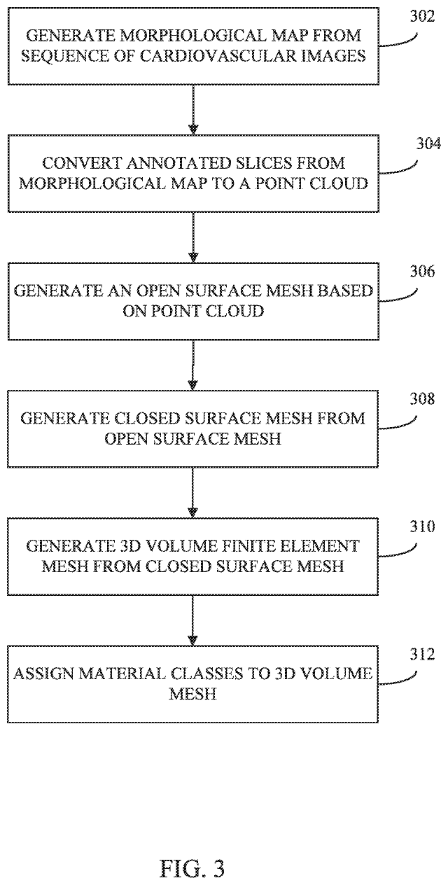

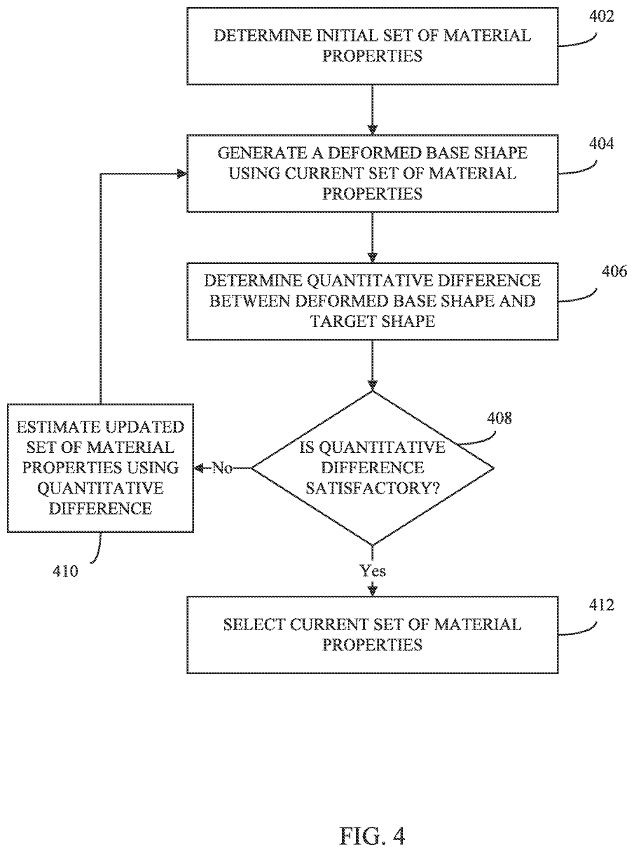

The pre-processing module 104 may be configured to convert each sequence of cardiovascular images to a three-dimensional (3D) volume finite element (FE) mesh with heterogenous material distribution as discussed further below with respect to FIGS. 2 and 3. Each 3D FE mesh can represent a shape or geometry of, for example, a vessel. For example, a first 3D FE mesh generated using a first sequence of cardiovascular images may represent a base shape or geometry and the second 3D FE mesh generated using the second sequence of cardiovascular images may represent a target shape. In some embodiments, the two generated 3D FE meshes may be stored in data storage such as, for example, data storage 114. The two 3D FE meses generated by the pre-processing module 104 (e.g., a base shape and a target shape) may be provided as an input to the optimizer 106 which is configured to determine or recover one or more material properties of at least one tissue in the region of interest based on the two 3D FE meshes and the set of pressure data for each sequence of cardiovascular images. As discussed further below with respect to FIGS. 2 and 4, in some embodiments, the optimizer 106 implements an optimization process that includes inverse FE simulations and an objective function that incorporates micro-morphological information in the form of interfaces between tissues (e.g., intra-plaque interfaces) in the region of interest. The optimizer 106 can generate an output 108 including one or more material properties of at least one tissue in the region of interest. In some embodiments, the determined material properties (or parameters) can be linear elastic material properties or nonlinear hyperelastic material properties. These material properties may include, for example, Young's modulus (i.e., modulus of elasticity), bulk modulus, shear modulus, Poisson's ratio, or Yeoh material parameters (e.g., C₁₀ and C₂₀). The determined material properties (or parameters) 108 may be displayed on a display 112. The determined material properties may also be stored in data storage, for example, data storage 114.

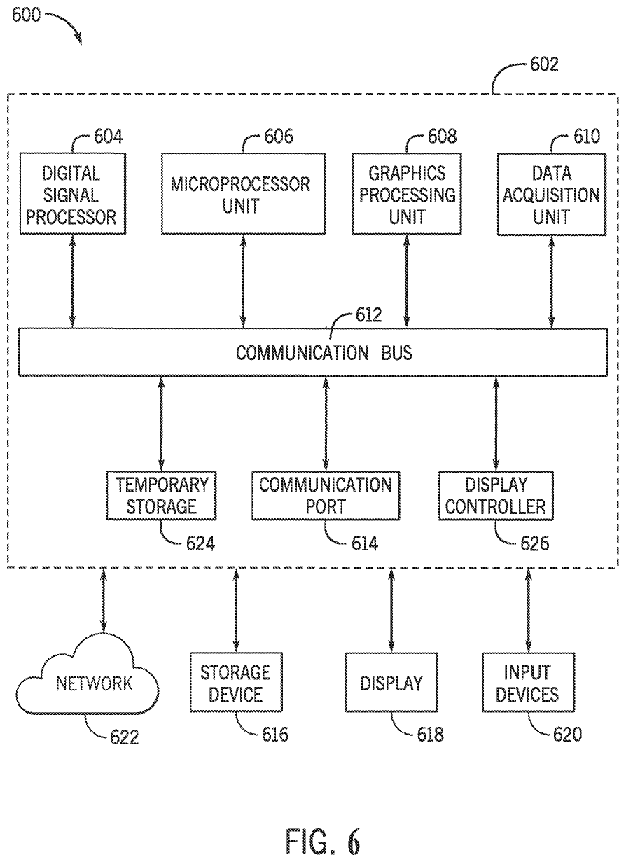

In some embodiments, the pre-processing module 104 and the optimizer 106 may be implemented on one or more processors (or processor devices) of a computer system (e.g., the example computer system 600 shown in FIG. 6) such as, for example, any general-purpose computing system or device, such as a personal computer, workstation, cellular phone, smartphone, laptop, tablet, or the like. As such, the computer system may include any suitable hardware and components designed or capable of carrying out a variety of processing and control tasks, including steps for receiving sequences of cardiovascular images of the subject and associated sets of pressure data 102, implementing pre-processing module 104, implementing optimizer 106, providing determined material properties 108 to a display 112 or storing the determined material properties 108 in data storage 114. For example, the computer system may include a programmable processor or combination of programmable processors, such as central processing units (CPUs), graphics processing units (GPUs), and the like. In some implementations, the one or more processor of the computer system may be configured to execute instructions stored in a non-transitory computer readable-media. In this regard, the computer system may be any device or system designed to integrate a variety of software, hardware, capabilities and functionalities. Alternatively, and by way of particular configurations and programming, the computer system may be a special-purpose system or device. For instance, such special-purpose system or device may include one or more dedicated processing units or modules that may be configured (e.g., hardwired, or pre-programmed) to carry out steps, in accordance with aspects of the present disclosure.

FIG. 2 illustrates a method for determining material properties of a plurality of tissues in a subject in accordance with an embodiment. The process illustrated in FIG. 2 is described below as being carried out by the system 100 for determining material properties of a plurality of tissues in a subject as illustrated in FIG. 1. Although the blocks of the process are illustrated in a particular order, in some embodiments, one or more of the blocks may be executed partially or entirely in parallel, may be executed in a different order than illustrated in FIG. 2, or may be bypassed.

At block 202, a first sequence of cardiovascular images of a region of interest and a first set of pressure data associated with the first sequence of cardiovascular images may be received from, for example, data storage 110 of system 100, data storage of an imaging system (e.g., an OCT imaging system), or data storage of other computer systems. At block 204, a second sequence of cardiovascular images of the region of interest and a second set of pressure data associated with the second sequence of cardiovascular images may be received from, for example, data storage 110 of system 100, data storage of an imaging system (e.g., an OCT imaging system), or data storage of other computer systems. The first sequence and second sequence of cardiovascular images can be acquired from a region of interest of a subject that includes a plurality of tissues. For example, in some embodiments, the plurality of tissues may include tissue types such as artery, fibrous, lipid, calcified, mixed, and healthy wall tissue. The first and second sequence of cardiovascular images may be acquired using known cardiovascular imaging techniques. In some embodiments, the first and second sequence of cardiovascular images can be acquired using intravascular imaging techniques, for example, OCT and IVUS, and acquired using known imaging systems (e.g., OCT or IVUS imaging systems). Accordingly, each sequence of cardiovascular images may be acquired using, for example, a pullback or tomographic acquisition. As discussed above, in some embodiments, the first and second sequence of cardiovascular images may be acquired using other cardiovascular imaging techniques such as MRI and CT. The first and second sequence of cardiovascular images may be acquired using the same or different cardiovascular imaging techniques.

The first and second sequence of cardiovascular images can provide, for example, three-dimensional (3D) information on spatial tissue distribution. In addition, the first sequence of cardiovascular images corresponds to or is associated with a first set of pressure data and the second sequence of cardiovascular images corresponds to or is associated with a second set of pressure data. For example, each sequence of cardiovascular images may be acquired at a constant or varying intraluminal pressure that can be measured during the image acquisition. Each of the two sequences of cardiovascular images can be acquired at different pressures. As discussed above, in some embodiments, the first and second set of pressure data may be measured using various apparatus and techniques such as, for example, a manometer, the imaging system or apparatus itself (for example, an imaging catheter with a built-in pressure transducer or an imaging catheter that includes materials of known stiffness), or a contrast or flush injector used for estimating the pressure during the imaging process, for example, a liquid contrast/flush.

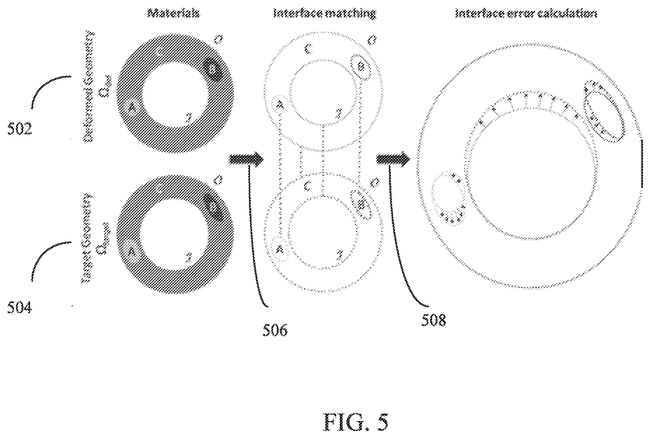

At block 206, the first sequence of cardiovascular images is preprocessed (e.g., using pre-processing module 104) to transform the first sequence of cardiovascular images to a first 3D finite element (FE) mesh with heterogeneous material distribution. At block 208, the second sequence of cardiovascular images is preprocessed (e.g., using pre-processing module 104) to transform the second sequence of cardiovascular images to a second 3D finite element (FE) mesh with heterogeneous material distribution. The first and second 3D FE mesh may include the same plurality of tissue types (e.g., artery, fibrous, lipid, calcified, mixed, and healthy wall tissue) as the first and second sequence of cardiovascular images, respectively. Alternatively, in some embodiments, the tissue types in the first and second FE meshes may be grouped into fewer heterogeneous classes of tissue types (e.g., resulting in less granular tissue type classes or groups). An example method for generating a 3D volume FE mesh with heterogeneous material distribution is described below with respect to FIG. 3. At block 210, an iterative optimization process may be performed (e.g., by optimizer 106) using the first and second 3D FE meshes as well as the interfaces between the plurality of tissues in the region of interest to determine one or more material properties of at least one of the plurality of tissues. For example, the optimization process may advantageously incorporate micro-morphological information in the form of interfaces between tissues (e.g., intra-plaque interfaces) into an objective function for the optimization process. In addition, in some embodiments, the optimization process can include an inverse FE process (e.g., FE simulation) in each iteration. Inverse FE modeling can recover material properties from known displacements and loading conditions. These methods utilize iterative rounds of FE simulations, where local material parameters are continuously tuned to minimize a predefined objective function to replicate experimentally measured displacements or geometric states. An example optimization method using an inverse 3D FE process and incorporating micro-morphological information is described below with respect to FIGS. 4 and 5. At block 212, the determined or recovered one or more material properties (or parameters) for at least one of the plurality of tissues may be displayed on a display 112 and/or stored in, for example, data storage 114 of system 100.

FIG. 3 illustrates a method for generating a three-dimensional (3D) finite element (FE) mesh based on a set of cardiovascular images of a subject in accordance with an embodiment. At block 302, a morphological map (or characterized image) may be generated from an acquired sequence of cardiovascular images (e.g., the first or second sequence of cardiovascular images received at blocks 202 and 204 of FIG. 2, respectively) of a region of interest in a subject and at block 304, the annotated slices of the morphological map may be converted to (or used to generate) a point cloud using known methods. For example, the information contained in the morphological map (or characterized image) may be extracted and represented in the form of a point cloud. As discussed above, the region of interest can have a plurality of tissues. In the morphological map, the plurality of tissue types in the region of interest can be annotated resulting in, for example, a set of annotated slices. In some embodiments, the morphological map may be generated using known automated annotation algorithms including, for example, deep learning methods (e.g., neural networks). An example method for morphological map generation is described in U.S. Pat. No. 11,122,981, “Arterial Wall Characterization in Optical Coherence Tomography Imaging,” herein incorporated by reference in its entirety. In some embodiments, for blocks 302 and 304, inner and outer borders of a vessel wall imaged in the sequence of cardiovascular images can be identified and fit with a smooth, continuous surface in 3D. The resulting region of interest may then be characterized with a validated deep learning method for classifying tissue micromorphology in cardiovascular images (e.g., OCT images), which automatically annotated frames with the spatial distribution of non-pathological and diseased (calcified, lipid, fibrotic, or mixed) tissue. In some embodiments, a subset of the total acquisition length may be taken to exclude low-quality images and/or reduce computational costs. The generated point cloud set may consist of pixel coordinates and corresponding tissue labels from the selected segment.

At block 306, the point cloud may be used to generate an open surface mesh using known methods. At block 308, a closed surface mesh may be generated from the open surface mesh, by, for example, connecting the open surface mesh at each end to form the closed surface mesh. The closed surface mesh may be generated using known methods. For example, in some embodiments, the points representing the inner and outer surfaces of the imaged vessel may be converted into triangulated meshes by Poisson surface reconstruction, then connected at each end to form a closed surface mesh. At block 310, a 3D volume finite element (FE) mesh with heterogeneous material distribution is generated using the closed surface mesh, for example, the closed surface mesh may be used as a scaffold to generate the 3D volume FE mesh. In some embodiments, the closed surface mesh may be converted into a 3D volume FE mesh of tetrahedral elements. In addition, at block 310, each element in the 3D volume FE mesh can be assigned to a material class based on the morphological annotations. For example, each element in the mesh may be assigned to the material class associated with the point in the annotated OCT dataset closest to its centroid. The resulting 3D tetrahedral FE mesh (δ)—with heterogeneous material distribution—can represent the transformation of visually-encoded data captured during clinical imaging into a discretized volumetric model amenable to structural simulation. In some embodiments, the first 3D FE mesh generated for the first sequence of cardiovascular images (e.g., as discussed above in block 202 of FIG. 2) associated with the first set of pressure data, P_{base}, may represent a base shape or geometry, δ_{base}, and the second 3D FE mesh generated for the second sequence of cardiovascular images (e.g., as discussed above in block 204 of FIG. 2) associated with the second set of pressure data, P_{target}, may represent a target shape or geometry, δ_{target}. In another embodiment, for the first 3D FE mesh generating using the first sequence of cardiovascular images (e.g., as discussed above with respect to block 202 of FIG. 2) associated with the first set of pressure data, P_{base}, known methods may be used to estimate a zero-pressure state of the imaged tissue, and the resulting zero-pressure geometry may represent a base shape or geometry, δ_{base}

As mentioned above, an iterative optimization process may be performed (e.g., by optimizer 106) using the generated first and second 3D meshes as well as interfaces between the plurality of tissues in the region of interest to determine one or more material properties of at least one of the plurality of tissues, for example, material parameters of multiple plaque components. As discussed above, first and second sequences of cardiovascular images (e.g., OCT images from pullbacks) acquired at two different pressures, P_{base} and P_{target}, can be converted into two 3D FE meshes, δ_{base} and δ_{target}, respectively, with heterogeneous material distributions. FIG. 4 illustrates an optimization method using an inverse 3D FE process in accordance with an embodiment. In some embodiments, the optimization method may be configured to utilize the 3D FE meshes (or models), δ_{base} and δ_{target}, to recover a vector of material properties ( [symbol] *) that results in observed displacements between the two models. In some embodiments, the optimization process is configured to minimize an objective function (δ).

At block 402, an initial set of material properties is determined for the iterative optimization process. In some embodiments, a multi-objective genetic optimization algorithm (e.g., the Non-dominated Sorting Genetic Algorithm (NSGA-II)) may first be used to identify a global region within which the optimal material parameter (or property) set is likely to exist. In some embodiments, in an example using NSGA-II, an initial population of parameters may be generated using a space-filling Latin Hypercube Sampling method. This population may then be propagated over several generations, with the fittest individuals being chosen to stochastically exchange parameters with each other. Once a narrowed region of interest has been identified by the multi-objective genetic optimization algorithm (e.g., the NSGA-II algorithm), an evolved single objective, sequential quadratic programming algorithm (e.g., NLPQLP) may be employed to determine an initial or starting parameter vector for the optimization method. In some embodiments, the individual yielding the minimum sum of all interface errors (δ_{SO}ⁱ, discussed further below) across all generations of the multi-objective genetic optimization algorithm (e.g., NSGA-II) run can be chosen as the starting parameter vector. In an example, the NLPOLP algorithm uses forward finite differences to evaluate the gradient at a given point; if there are n parameters to be optimized, each function evaluation requires n+1 simulations, one for the point itself and n simulations for the n-dimensional gradient evaluation. The algorithm stops when the difference between successive objectives drops below a given threshold or once it reaches a predefined limit on number of function evaluations.

At block 404, a deformed base shape, (δ_{def}), may be generated using the current set of material properties ( [symbol] ⁽ⁱ⁾), for example, for the first iteration, the initial or starting parameter vector determined at block 402. In some embodiments, for each iteration i, an FE simulation ( [symbol] ) of the deformed base shape (δ_{def}) may be performed by applying the pressure differential, ΔP=P_{target}−P_{base}, using the current material parameter vector ( [symbol] ⁽ⁱ⁾). P_{base} and P_{target} represent the different intraluminal pressure states at which the images corresponding to δ_{base} and δ_{target} were acquired. For example, during the optimization process, the base shape (δ_{base}) can be iteratively simulated into the deformed shape (δ_{def}), using the current vector of assigned material parameters ( [symbol] ⁽ⁱ⁾) for iteration i:

δ_{def}⁽ⁱ⁾⁼ [symbol] ^{(δ}_{base},ΔP, [symbol] ⁽ⁱ⁾); Eqn. 1

In some embodiments, the optimization method may consider zero-pressure (i.e., unloaded) geometry in addition to the imaged geometry. In some embodiments, zero-pressure geometry may be estimated prior to the optimization process using known methods which do not rely on material properties. In such embodiments, the zero-pressure state of the imaged tissue may be used as δ_{base}. Accordingly, the pressure differential in the intraluminal pressure state at which the images corresponding to δ_{target} may be acquired as: ΔP=P_{target}. Alternatively, the imaged state may be used as δ_{base}, and the zero-pressure state may be used to estimate an initial stress distribution in the a δ_{base} model prior to loading by ΔP=P_{target}−P_{base} to determine the deformed base shape (δ_{def}). In some embodiments, known methods for estimating zero-pressure geometry which use the current set of material properties (e.g., the inverse FE) may be incorporated into the iterative process for generating the deformed base shape. In such embodiments, the simulation may occur in a multi-step process whereby the zero-pressure geometry is first estimated using known methods, and the deformed geometry is then simulated using the zero-pressure geometry or the imaged base geometry with imposed pre-stress implied by the unloaded geometry. In another embodiment, zero-pressure geometry may be estimated using known methods for both sets of geometries, with one serving as δ_{def} and the other as δ_{target} for the purpose of quantifying shape differences to perform material property optimization.

In some embodiments, P_{base} and P_{target} may be constant values or may be vectors or time-series of pressure values. When pressures are not constant, the deformed shape (δ_{def}) may be estimated by performing a static FE simulation ( [symbol] ) with a spatially-varying pressure corresponding to the pressures at various regions of the tissue were imaged, or by a dynamic FE simulation ( [symbol] ) with time-varying pressure. In an embodiment using dynamic FE simulation ( [symbol] ) with time-varying pressure, the deformed shape (δ_{def}) may be estimated by extracting geometry (e.g., node positions) from various time points throughout the simulation corresponding to the time or pressure at which the various regions of tissue were imaged.

At block 406, for each iteration i, a quantitative difference between the deformed base shape, δ_{def}, and the target shape, δ_{target}, may then be computed by, for example, a difference operator ( [symbol] ) and represented by the objective function:

δ⁽ⁱ⁾= [symbol] (δ_{def}⁽ⁱ⁾,δ_{target}); Eqn. 2

As mentioned above, in some embodiments, micro-morphological information in the form of interfaces between tissue regions may advantageously also be incorporated in the formulation of the objective function (δ). In some embodiments, with node sets (contained in an array [symbol] ) being defined for each material region and for the inner and outer surfaces, an interface can be defined as the set of common nodes between two node sets. For each node rⱼ^{target|X,Y} in the target geometry belonging to an interface between node sets X and Y (X∩Y, X,Yε [symbol] :Y≠X), the Euclidean distance to the nearest node rₖ^{def|X,Y} belonging to the corresponding interface in the deformed base geometry, δ_{def}, may be calculated. The average of all such distances may comprise the interface error, ε^{X,Y}:

$${ϵ}^{X , Y} = \frac{1}{{N}^{target \vert X , Y}} {\sum }_{j = 1}^{{N}^{target \vert X , Y}} {min}_{k ∈ \{ 1 , … , {N}^{def \vert X , Y} \}} \vert {r}_{j}^{target \vert X , Y} - {r}_{k}^{def \vert X , Y} \qquad Eqn . 3$$

where N^{def|X,Y} and N^{target|X,Y} are the number of nodes in the interfaces of the deformed (δ_{def}) and target geometries, δ_{target}, respectively.

FIG. 5 is a diagram illustrating an example difference operator ( [symbol] ) quantifying multiple feature changes for use in an objective function of the optimization method in FIG. 4 in accordance with an embodiment. To illustrate the above described formulation, FIG. 5 shows a simplified scenario with multiple plaque components. In this example, the deformed geometry 502 (δ_{def}) and the target geometry 504 (δ_{target}) have five node sets contained in list [symbol] , where [symbol] ={A, B, C, [symbol] , [symbol] }. Three of the node sets represent material regions (A, B, and C), and the other two include the inner and outer surfaces ( [symbol] and [symbol] ). This yields four corresponding interfaces on the deformed (solid) 502 and target (dashed) 504 geometries in total: two between the material region C and the inner and outer surfaces (A∩C and B∩C), and two between the material region C and the inner and outer surfaces ( [symbol] ∩C and [symbol] ∩C. In this example, there are consequently four interface errors to be calculated: ε^{A,C},ε^{B,C}, [symbol] and [symbol] . In some embodiments, the corresponding interfaces on the deformed (solid) 502 and target (dashed) 504 geometries may be compared by calculating the average Euclidean distance between their nodes (as indicated by arrows 506, 508), based on a nearest neighbor search.

Referring again to FIG. 4, the objective function quantifying the discrepancy or difference between the two geometries, δ_{def} and δ_{target}, at each iteration, i, may then be formulated as a function of these individual interface errors. In a multi-objective optimization algorithm, the objective function may be defined as the vector of all the unweighted interface errors:

δ_{MO}ⁱ={ε^{X,Y}∀X,Y ε [symbol] :Y≠X} Eqn. 4

where δ_{MO}ⁱ is the multi-objective vector. In a single-objective optimization, the objective, δ_{SO}ⁱ, must be a single scalar value and may be defined as the sum of all interface errors:

δ_{SO}ⁱ= [symbol] ε^{X,Y; X,Yε} [symbol] ^{:Y≠X}

This objective function, through the matching of interfaces, allows for the recovery of material parameters for multiple different material sets, using acquisitions at only two states.

At block 408, it is determined whether the quantitative difference, δ⁽ⁱ⁾, is sufficiently small, for example, if the current set of material parameters minimizes the objective function, δ, as given by:

$${𝕐}^{*} = arg {min}_{𝕐} δ \qquad Eqn . 6$$

where

$$arg {min}_{𝕐}$$

δ refers to the set of parameters [symbol] that results in the minimum of value of δ. If the quantitative difference is satisfactory, the current set of material properties can be selected at block 412 and provided as an output (e.g., of optimizer 106 shown in FIG. 1). Accordingly, the goal of the inverse FE method of the optimization process can be to find the vector of optimal material parameters, [symbol] *, such that the base and target geometries, δ_{base} and δ_{target}, are matched under the given loading condition, ΔP.

If the quantitative difference is not satisfactory at block 408, for each iteration i, the quantitative difference (δ⁽ⁱ⁾) determined at block 406 may be used at block 410 to estimate and refine a subsequent estimate of a set of material parameters (or properties), [symbol] ⁽ⁱ⁺¹⁾. The process then returns to block 404 for the next iteration.

In some embodiments, the material characterization system and method relies neither on elastography measurements nor on several (>2) sequences of image acquisitions. By blending morphological assessment with displacement observations, two intravascular image acquisitions with pressure data can provide sufficient information to estimate the multiparameter material properties (e.g., linear or nonlinear) of, for example, multiple plaque components. In some embodiments of the disclosed mechanical characterization system and method, only the initial and final states were compared, alleviating the need for full displacement maps. Specifically, displacements of, for example, intraplaque features can be tracked such that a constrained number of constitutive parameters defined the loading response of the entire vessel. By tracking intraplaque interfaces in addition to the inner and outer surfaces, a richer set of data can be extracted from each acquisition, thereby enabling the recovery of a higher number of material parameters (e.g., eight).

The reliance on 3D morphological information in the disclosed mechanical characterization system and method can obviate the need for additional image processing including recovery of local strain state (as in elastography imaging). This can not only circumvent common sources of error, but can also make the approach readily applicable across imaging modalities, some of which may not possess an elastography module or developed capabilities. In addition, the use of only two displacement states (baseline and target) acquired at physiological extrema (peak systole vs. late diastole) may be sufficient to fully recover underlying constitutive tissue properties. As such, the disclosed mechanical characterization system and method may avoid any external intravascular pressure elevation, where instead full material recovery is permitted simply using physiological pressure variations observed during normal homeostatic cardiovascular performance.

FIG. 6 is a block diagram of an example computer system in accordance with an embodiment. Computer system 600 may be used to implement the systems and methods described herein. In some embodiments, the computer system 600 may be a workstation, a notebook computer, a tablet device, a mobile device, a multimedia device, a network server, a mainframe, one or more controllers, one or more microcontrollers, or any other general-purpose or application-specific computing device. The computer system 600 may operate autonomously or semi-autonomously, or may read executable software instructions from the memory or storage device 616 or a computer-readable medium (e.g., a hard drive, a CD-ROM, flash memory), or may receive instructions via the input device 620 from a user, or any other source logically connected to a computer or device, such as another networked computer or server. Thus, in some embodiments, the computer system 600 can also include any suitable device for reading computer-readable storage media.

Data, such as data acquired with an imaging system (e.g., an OCT imaging system, an intravascular ultrasound (IVUS) imaging system, etc.) may be provided to the computer system 600 from a data storage device 616, and these data are received in a processing unit 602. In some embodiment, the processing unit 602 includes one or more processors. For example, the processing unit 602 may include one or more of a digital signal processor (DSP) 604, a microprocessor unit (MPU) 606, and a graphics processing unit (GPU) 608. The processing unit 602 also includes a data acquisition unit 610 that is configured to electronically receive data to be processed. The DSP 604, MPU 606, GPU 608, and data acquisition unit 610 are all coupled to a communication bus 612. The communication bus 612 may be, for example, a group of wires, or a hardware used for switching data between the peripherals or between any component in the processing unit 602.

The processing unit 602 may also include a communication port 614 in electronic communication with other devices, which may include a storage device 616, a display 618, and one or more input devices 620. Examples of an input device 620 include, but are not limited to, a keyboard, a mouse, and a touch screen through which a user can provide an input. The storage device 616 may be configured to store data, which may include data such as, for example, acquired sequences of cardiovascular images and pressure data, whether these data are provided to, or processed by, the processing unit 602. The display 618 (e.g., display 112 in FIG. 1) may be used to display images and other information, such as the sequences of cardiovascular images, sets of pressure data, recovered material properties, morphological maps, 3D FE meshes, and so on.

The processing unit 602 can also be in electronic communication with a network 622 to transmit and receive data and other information. The communication port 614 can also be coupled to the processing unit 602 through a switched central resource, for example the communication bus 612. The processing unit can also include temporary storage 624 and a display controller 626. The temporary storage 624 is configured to store temporary information. For example, the temporary storage 624 can be a random access memory.

Computer-executable instructions for determining material properties of a plurality of tissues in a subject according to the above-described methods may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by interne or other computer network form of access.

The present disclosure has been described in terms of one or more preferred embodiments, and it should be appreciated that many equivalents, alternatives, variations, and modifications, aside from those expressly stated, are possible and within the scope of the invention.

1. A method for determining material properties of a plurality of tissues in a subject, the method comprising:
receiving a first sequence of cardiovascular images of a region of interest of the subject and a first set of pressure data associated with the first sequence of cardiovascular images, wherein the region of interest includes a plurality of tissues;
receiving a second sequence of cardiovascular images of the region of interest of the subject and a second set of pressure data associated with the second sequence of cardiovascular images, wherein the region of interest includes the plurality of tissues;
transforming, using one or more processors, the first sequence of cardiovascular images to a first three-dimensional (3D) finite element (FE) mesh with heterogeneous material distribution;
transforming, using the one or more processors, the second sequence of cardiovascular images to a second 3D FE mesh with heterogeneous material distribution;
performing, using the one or more processors, an iterative optimization process on the first and second 3D FE meshes to determine one or more material properties of at least one of the plurality of tissues, wherein the iterative optimization process utilizes interfaces between the plurality of tissues.

2. The method according to claim 1, wherein the first sequence of cardiovascular images and the second sequence of cardiovascular images are acquired using intravascular imaging.

3. The method according to claim 2, wherein the first sequence of cardiovascular images and the second sequence of cardiovascular images are optical coherence tomography (OCT) images.

4. The method according to claim 1, wherein transforming the first sequence of cardiovascular images to a first 3D FE mesh comprises generating characterized images for the first sequence of cardiovascular images and wherein transforming the second sequence of cardiovascular image to a second 3D FE mesh comprises generating characterized images for the second sequence of cardiovascular images.

5. The method according to claim 4, wherein transforming the first sequence of cardiovascular images to a first 3D FE mesh further comprises converting the characterized images for the first sequence of cardiovascular images to a point cloud and wherein transforming the second sequence of cardiovascular image to a second 3D FE mesh further comprises converting the characterized images for the second sequence of cardiovascular images to a point cloud.

6. The method according to claim 1, wherein the first 3D FE mesh represents a base shape and the second 3D FE mesh represents a target shape.

7. The method according to claim 1, wherein the first 3D FE mesh is used to derive a base shape and the second 3D FE mesh is used to derive a target shape.

8. The method according to claim 6, wherein the iterative optimization process is configured to determine a vector of material properties that results in an observed displacement between the base shape and the target shape.

9. The method according to claim 6, further comprising generating a deformed base shape from the base shape, wherein the iterative optimization process minimizes an objective function which quantifies a difference between the deformed base shape and the target shape.

10. The method according to claim 1, wherein the iterative optimization process minimizes an objective function which quantifies a distance between corresponding interfaces between the plurality of tissues in the first 3D FE mesh and the second 3D FE mesh.

11. The method according to claim 1, wherein the plurality of tissues includes components of arterial plaque.

12. The method according to claim 1, wherein the one or more material properties includes linear elastic material parameters.

13. The method according to claim 1, wherein the one or more material properties includes nonlinear hyperelastic material parameters.

14. A system for determining material properties of a plurality of tissues in a subject, the system comprising:
an input configured to receive a first sequence of cardiovascular images of a region of interest of the subject and a first set of pressure data associated with the first sequence of cardiovascular images, and configured to receive a second sequence of cardiovascular images of the region of interest of the subject and a second set of pressure data associated with the second sequence of cardiovascular images, wherein the region of interest includes a plurality of tissues;
a pre-processing module configured to transform the first sequence of cardiovascular images to a first three-dimensional (3D) finite element (FE) mesh with heterogeneous material distribution, and configured to transform the second sequence of cardiovascular images to a second 3D FE mesh with heterogeneous material distribution; and
an optimizer configured to perform an iterative optimization process on the first and second 3D FE meshes to determine one or more material properties of at least one of the plurality of tissues, wherein the iterative optimization process utilizes interfaces between the plurality of tissues.

15. The system according to claim 14, further comprising a memory coupled to the optimizer, the memory configured to store the one or more material properties.

16. The system according to claim 14, further comprising a display coupled to the optimizer, the display configured to display the one or more material properties.

17. The system according to claim 14, wherein the first sequence of cardiovascular images and the second sequence of cardiovascular images are acquired using intravascular imaging.

18. The system according to claim 17, wherein the first sequence of cardiovascular images and the second sequence of cardiovascular images are optical coherence tomography (OCT) images.

19. The system according to claim 14, wherein transforming the first sequence of cardiovascular images to a first 3D FE mesh comprises generating characterized images for the first sequence of cardiovascular images and wherein transforming the second sequence of cardiovascular image to a second 3D FE mesh comprises generating characterized images for the second sequence of cardiovascular images.

20. The system according to claim 14, wherein the first 3D FE mesh represents a base shape and the second 3D FE mesh represents a target shape.

21. The system according to claim 14, wherein the first 3D FE mesh is used to derive a base shape and the second 3D FE mesh is used to derive a target shape.

22. The system according to claim 20, wherein the iterative optimization process is configured to determine a vector of material properties that results in an observed displacement between the base shape and the target shape.

23. The system according to claim 20, wherein the optimizer is further configured to generate a deformed base shape from the base shape and the iterative optimization process minimizes an objective function which quantifies a difference between the deformed base shape and the target shape.

24. The system according to claim 14, wherein the iterative optimization process minimizes an objective function which quantifies a distance between corresponding interfaces between the plurality of tissues in the first 3D FE mesh and the second 3D FE mesh.

25. The system according to claim 14, wherein the plurality of tissues includes components of arterial plaque.

26. The system according to claim 14, wherein the one or more material properties includes linear elastic material parameters.

27. The system according to claim 14, wherein the one or more material properties includes nonlinear hyperelastic material parameters.
